(19) United States
(12) Patent Application Publication
(10) Pub. No.: US 2017/0326673 A1
(43) Pub. Date: Nov. 16, 2017

(54) WELDING ASSISTANCE DEVICE WITH A WELDING MASK HAVING A VELOCITY SENSOR

(71) Applicant: Nuovo Pignone Srl, Florence (IT)

(72) Inventors: Francescosaverio CHIARI, Florence (IT); Marco MAGNASCO, Massa (IT); Mario MILAZZO, Rocca Massima (IT); Cesare STEFANINI, Vicopisano (IT); Francesco INGLESE, Grottaminarda (IT)

(21) Appl. No.: 15/529,727

(22) PCT Filed: Nov. 20, 2015
(86) PCT No.: PCT/EP2015/077217

§ 371 Date: May 25, 2017

(30) Foreign Application Priority Data
Nov. 27, 2014 (IT) MI2014A002042

(51) Int. Cl. B23K 9/32; B23K 9/167; B23K 9/095; B23K 37/00; A61F 9/06

(52) U.S. Cl. CPC B23K 9/322; B23K 37/006; A61F 9/06; B23K 9/0956; B23K 9/167

(57) ABSTRACT

A welding assistance device comprises a welding mask, a welding velocity sensor attached on the welding mask and configured to detect a welding velocity; a visualization device attached to the welding mask and arranged to show a representation of the welding velocity and of consequent heat input to a welder.

### BACKGROUND

The subject matter of the present disclosure relates to a welding mask and an arc welding kit, namely a set of tools that is used to perform a manual arc welding operation.

A known arc welding kit comprises a welding mask and a welding tool. The welding tool comprises an electrode. During welding operations, an electric arc develops between the electrode and the welding area.

In a first type of arc welding, the SMAW (Shielded Metal Arc Welding), the electrode itself melts due to the heat developed by the electric arc, thus becoming the filler material in the weld. In a second type of arc welding, the TIG (Tungsten Inert Gas), the electrode is solid, and the filler material is provided separately.

With more detail, the kit comprises a set of sensors which can detect the main operating parameters of a welding process, namely the voltage (V), the current (A), the welding speed (W) and their combination to calculate the heat input. The welding mask can be provided with a display device so that these parameters can be shown to a welder, thereby providing him with a possibility of correcting the welding in real time. An example of this welding mask is the one shown in the document U.S. Pat. No. 6,242,711 B1.

A disadvantage of the known welding kit is that it merely provides the welder with the welding parameters. However, this does not guarantee that the welder is able to adapt and correct a welding that is being performed improperly. In other words, the welding operation itself still relies heavily on the manual skill of the operator. This is particularly true with respect to the welding voltage, since it is mainly determined by the distance of the electrode from the weld area.

### SUMMARY

An embodiment of the invention therefore relates to a welding assistance device. Such device comprises a welding mask and a welding velocity sensor attached on the welding mask. The welding velocity sensor is configured to detect a welding velocity. A visualization device is attached to the welding mask and is arranged to show a representation of the welding velocity and of consequent heat input to a welder.

In an embodiment, in this way the welder has a feedback on the welding velocity which, in addition to a constant current and to a stable voltage, greatly improves the overall quality of the weld allowing to respect the target heat input.

Optionally, the welding assistance device also comprises a control unit having a processing module which is configured to compute a velocity difference between the welding velocity and a target velocity value for the welding velocity. The processing module can also be configured to emit a velocity difference signal representing the result of the velocity difference. The visualization device is then configured to acquire the velocity difference signal and to show a representation of the velocity difference to a welder.

Another embodiment of the invention relates to an arc welding kit comprising the above described welding assistance device. The kit also comprises a welding tool configured to be held by a welder. The welding tool comprises an electrode. The welding tool also comprises a voltage sensor, which is configured to detect a welding voltage between the electrode and the weld area and to emit a voltage signal representing a value of said welding voltage.

Optionally, the processing module is configured to compute a voltage difference between the welding voltage and a target voltage. The processing module is also configured to emit a voltage difference signal representing the result of such voltage difference. The visualization device on the welding mask can also be configured to acquire the voltage difference signal and to show a representation of the voltage difference to a welder.

Optionally, the electrode is consumable for performing a SMAW weld.

Alternatively, the electrode is non-consumable, thereby enabling the welder to perform a TIG weld. In other words, in this case the welding tool is a TIG welding torch.

### BRIEF DESCRIPTION OF THE DRAWINGS

Further details and specific embodiments will refer to the attached drawings, in which:

FIG. 1 is a schematic representation of an arc welding kit according to an embodiment;

FIG. 2 is a side sectional view of a component of the kit of FIG. 1;

FIG. 3 is a front sectional view of the component of FIG. 2;

FIG. 4 is a side sectional view of a component of the kit of FIG. 1, according to a different embodiment;

FIG. 5 is a side sectional view of a second component of the kit of FIG. 1;

FIG. 6 is a top sectional view of the component of FIG. 5;

FIG. 6A is an enlarged view of a detail of FIG. 6;

FIG. 7 is a front sectional view of the component of FIGS. 5 and 6;

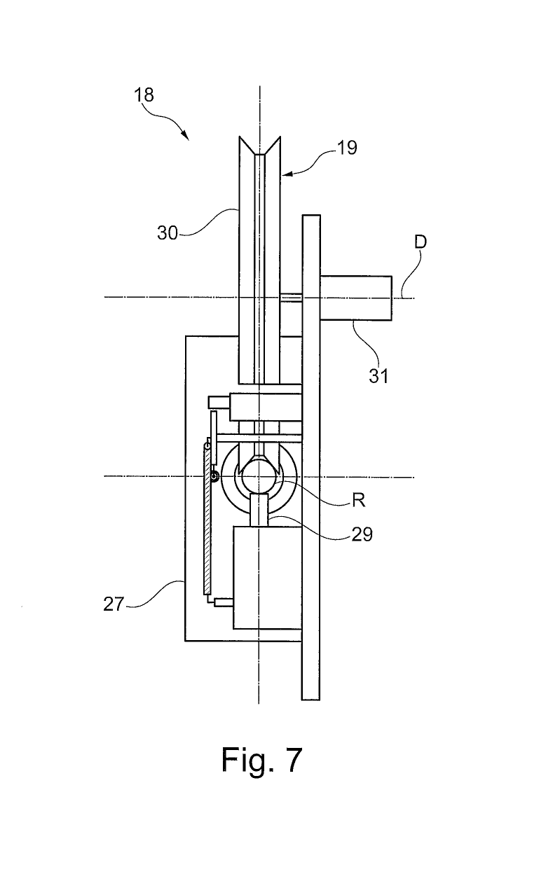

FIG. 8 is a front view of a welding assistance device according to an embodiment of the present invention; and

FIG. 9 is a schematic representation of the functioning of the kit of FIG. 1.

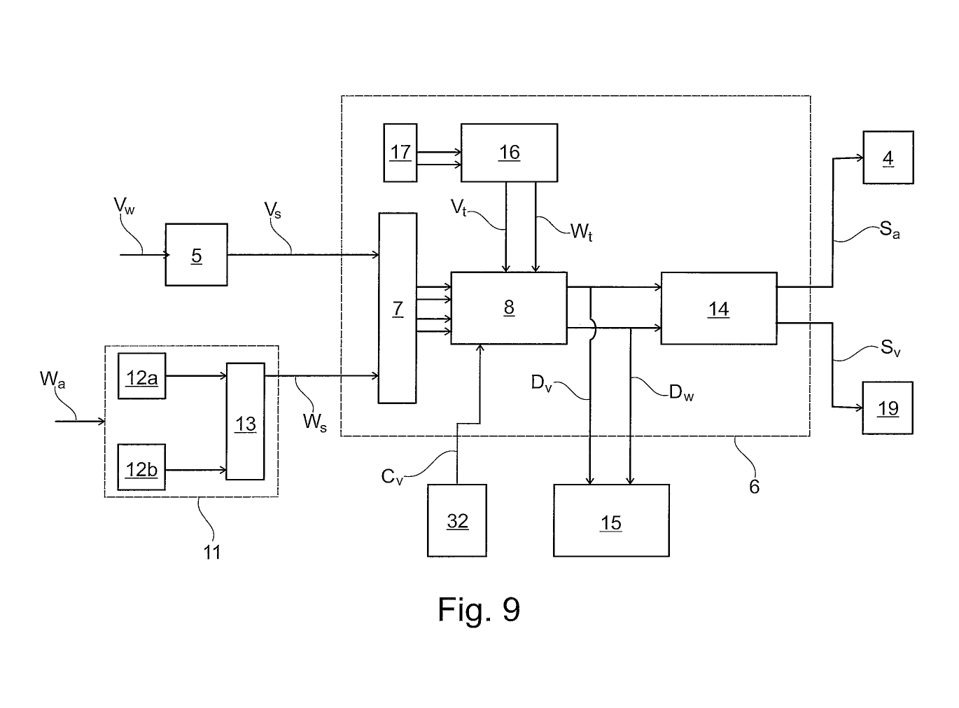

### DETAILED DESCRIPTION

The following description of exemplary embodiments refer to the accompanying drawings. The same reference numbers in different drawings identify the same or similar elements. The following detailed description does not limit the invention. Instead, the scope of the invention is defined by the appended claims.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

With reference to the attached drawings, with the number 1 is indicated an arc welding kit according to an embodiment of the present invention.

The welding kit 1 comprises a welding tool 2, which is configured to be held by a welder.

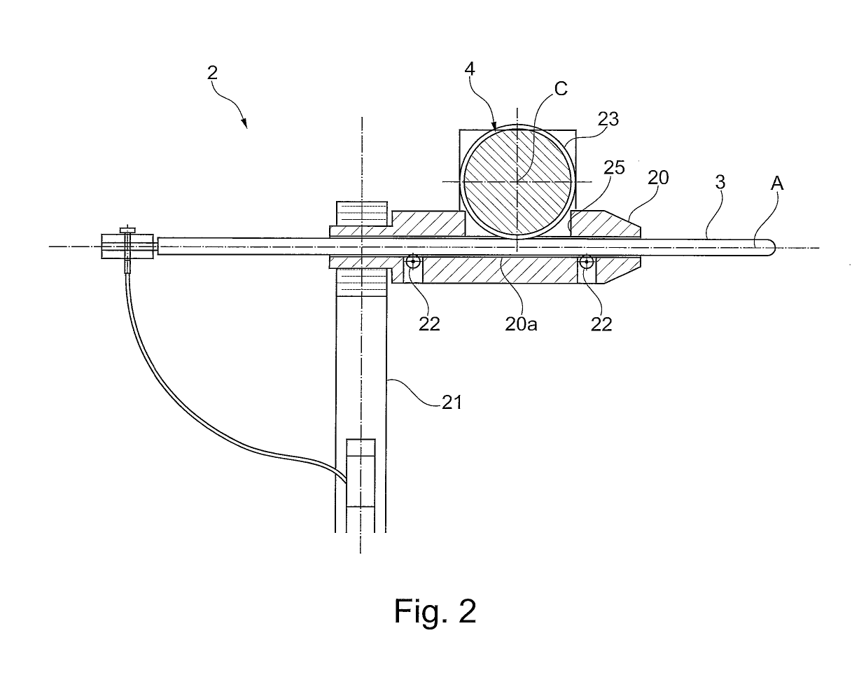

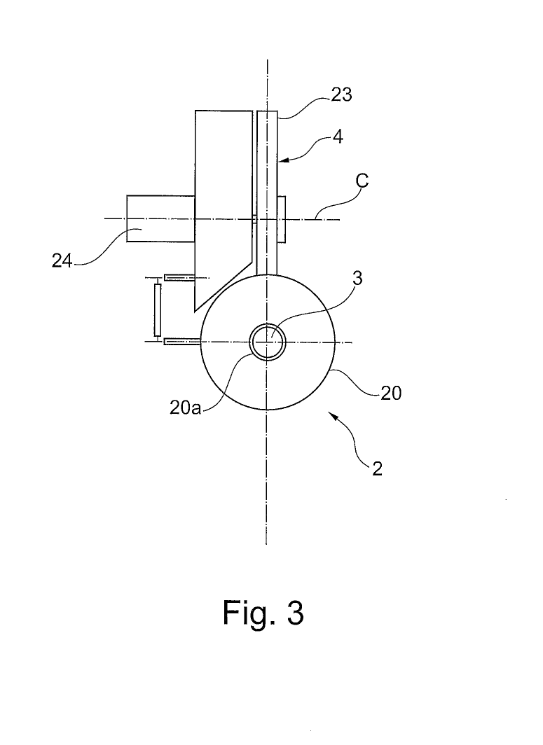

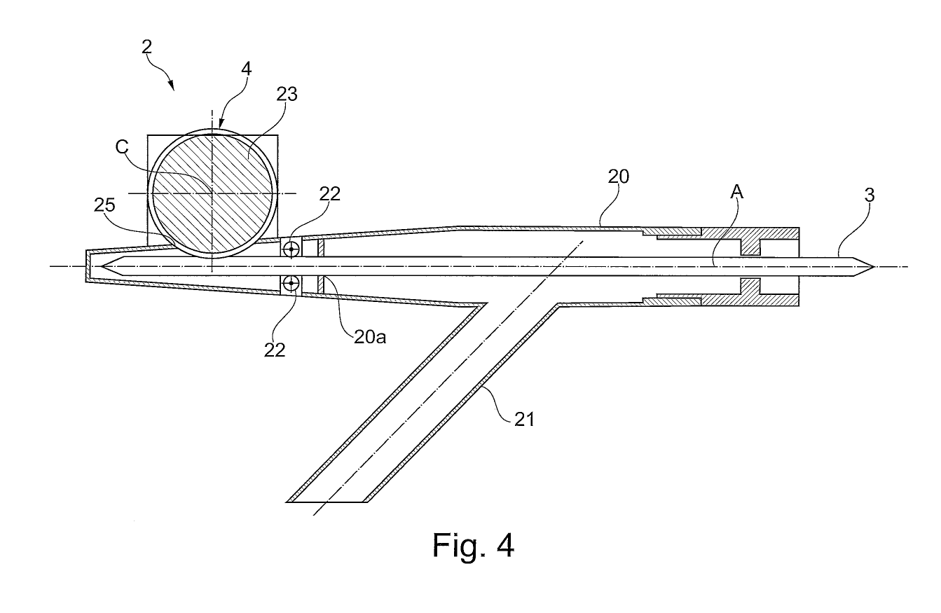

The welding tool 2 comprises an electrode 3. In a first embodiment, which is used to perform a SMAW (shielded metal arc weld), shown in FIGS. 2 and 3, the electrode 3 is consumable. In other words, in this embodiment the electrode 3 becomes the filler material of the weld. In another embodiment, shown in FIG. 4, the electrode 3 is non-consumable, thus it is used to perform a TIG (Tungsten Inert Gas) weld.

With additional details, the welding tool 2 comprises a main body 20, configured to support the electrode 3. The main body 20 is, in an embodiment, axial-symmetric, and develops mainly along a longitudinal axis “A”. A handle 21 for the welder supports the main body 20.

The main body 20 has a seat 20A in which the electrode 3 is installed. As shown in FIGS. 2 and 4, the welding tool 2 is provided with bearings 22, which are attached to the main body and located in proximity of the seat 20, so that they can support the electrode 3 and allow it to move forward and backward. In other words, the electrode 3 can move forward and backward inside the seat 20A by sliding on the bearings 22.

Also, the welding tool 2 comprises an adjusting device 4 associated with the electrode 3, in order to move the electrode forward/backward with respect to the main body 20. The adjusting device 4 comprises a wheel 23 having a central axis “C” disposed transversally and, in an embodiment, perpendicularly, to the longitudinal axis “A” of the electrode 3, which is parallel to the axis of the main body 20. Indeed, the main body 20 is provided with a port 25 in which the wheel 24 is inserted.

In operation, the rim of the wheel 23 is in contact with the electrode 3 so that the electrode 3 can be moved along the longitudinal axis “A” by a rotation of the wheel 23 along the central axis “C”. The adjusting device 4 also comprises a motor 24. Such motor 24 is, in an embodiment, electric, more particularly an electromagnetic motor, and is installed on the wheel 23 in order to actuate the wheel 23 and through it, the electrode 3.

With particular reference to the SMAW welding tool 2 of FIG. 2 please note that, in use, the wheel 23 advances overtime since the electrode 3 is consumed during welding. Therefore, the rotation speed of the motor 24 provides an overall forward movement to the electrode, and varies the rotation speed in order to adjust the distance of the tip of the electrode 3 as will be explained in a following part of the disclosure.

On the other hand, in the TIG welding tool 2 of FIG. 4 the electrode 3 is not consumed during welding. Therefore, the wheel 23 is moved only to adjust the distance of the electrode 3.

Also, in the embodiment of FIG. 4 a source of inert gas is present (not shown in the drawings) in order to shield the tip of the electrode 3 and the weld area from atmospheric oxygen. This source of inert gas is by itself known to the person skilled in the art, thus will not be described in detail.

The kit 1 comprises a voltage sensor 5 which is configured to detect a welding voltage “V_{w}” between the electrode 3 and the weld area, that is function of the distance between the end of the electrode facing the work piece and the weld area of the work piece. The voltage sensor 5 is also configured to emit a voltage signal “Vₛ”, which is representative of a value of the welding voltage “V_{w}”. Such voltage sensor 5 can be of any type known to the person skilled in the art, and therefore will not be described in detail.

The kit 1 also comprises a control unit 6. In the following part of the disclosure, the control unit 6 will be described by subdividing it into a plurality of modules. Such subdivision is done for ease of description only, and in no way, should be considered as reflecting the physical structure of the control unit 6 itself. Rather, each module can be implemented as an electronic circuit on a suitable hardware support, as a software routine, subroutine or library or as both. Each module may reside on a local unit or may be distributed over a network. Also, the modules can communicate with each other either via a suitable wired or wireless protocol.

The control unit 6 comprises a data acquisition module 7, which is configured to acquire the above-mentioned voltage signal “Vw”.

The control unit 6 also comprises a memory module 16, which is configured to store a target voltage value “Vt”.

The control unit 6 also comprises an input module 17 configured to set said target voltage value “Vt” in said memory module 16. In a particular embodiment of the invention, the input module 17 can be a QR code reader. In this way, the voltage “Vt”, as well as any other parameter related to the welding process, can be read by the input module 17 on a suitably encoded QR code.

The control unit 6 also comprises a processing module 8, which is configured to output an actuation signal “Sa” function of at least the voltage signal “Vs”. Also, the processing module 8 is configured to retrieve the target voltage value “Vt” and to compare it with the welding voltage value “Vw”. The actuation signal “Sa” is therefore at least in part directly proportional to the result of such comparison. With additional detail, the processing module 8 may be programmed with a PID (Proportional, Integral and Derivative) logic. Therefore, the actuation signal “Sa” may be the sum of a part directly proportional to the difference between “Vw” and Vt”, of a part proportional to the derivative of such difference and of a part proportional to the integral of such difference. Any possible combination can be used, depending on the chosen control strategy. The processing module 8 can also be configured to supply a voltage difference signal “Dv” representing the result of the difference between “Vw” and “Vt”.

The control unit 6 also comprises an actuation module 14 connected to the adjusting device 4. The actuation module 14 is configured to operate the adjusting device 4 as directed by the actuation signal “Sa”. In particular, the actuation module 14 operates the motor 24 which rotates the wheel 23. Optionally, the welding kit also comprises a welding mask 9. Such welding mask 9 is configured to be worn by a welder during a welding process as a standard safety mask.

In particular, the welding mask 9 comprises a darkened window 10 from which the welder may observe the welding process without being blinded by the intense light.

Additionally, the welding mask 9 is provided with a welding velocity sensor 11. The welding velocity sensor 11 is configured to detect a welding velocity “Wa”, and to emit a welding velocity signal “Ws” representing a value of the welding velocity “Wa”.

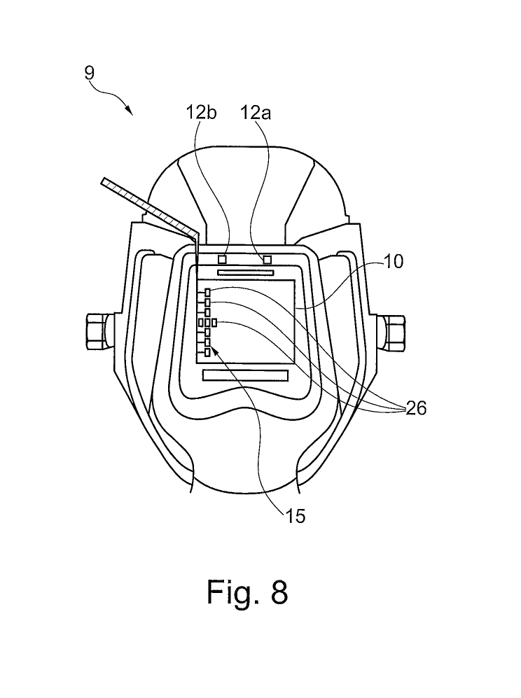

According to a preferred embodiment of the invention, the welding velocity sensor 11 comprises a first optical sensor 12A. The first optical sensor 12A is, in particular arranged so that, during welding operation it faces the weld area. As shown in FIG. 8, the first optical sensor is placed on the external surface of the welding mask 9, over the darkened window 10. The welding velocity sensor 11 also comprises a reference frame sensor 12B. This reference frame sensor 12B can be any kind of sensor which is able to detect a motion within a fixed frame of reference. For example, the reference frame sensor 12B can be an inertial sensor located on any point of the welding mask 9.

With more detail, in the embodiment shown in FIG. 8 the reference frame sensor 12B is a second optical sensor. The reference frame sensor 12B is therefore arranged to face a fixed reference scene in the environment, as for example the work piece part from the weld area, and placed more particularly beside the first optical sensor 12A. In an embodiment of the invention, the sensors 12A, 12B are imaging cameras.

With additional detail, the first optical sensor 12A is configured to detect the velocity of the welding pool relative to itself Also, the reference frame sensor 12B is configured to detect the velocity of the above mentioned fixed reference scene. According to one embodiment, the welding velocity sensor 11 also comprises a velocity computing module 13 which is configured to compute the welding velocity “Wa” as a difference between the velocities detected by the second 12B and the first optical sensor 12A. Alternatively, the first optical sensor 12A and reference frame sensor 12B both transmit the respective velocities to the control unit 6, in particular to the data acquisition module 14.

The processing module 8 is also configured to compute a velocity difference between the welding velocity “Wa” and a target velocity “Wt” value, said processing unit being configured to emit a velocity difference signal “Dw” representing the result of said velocity difference.

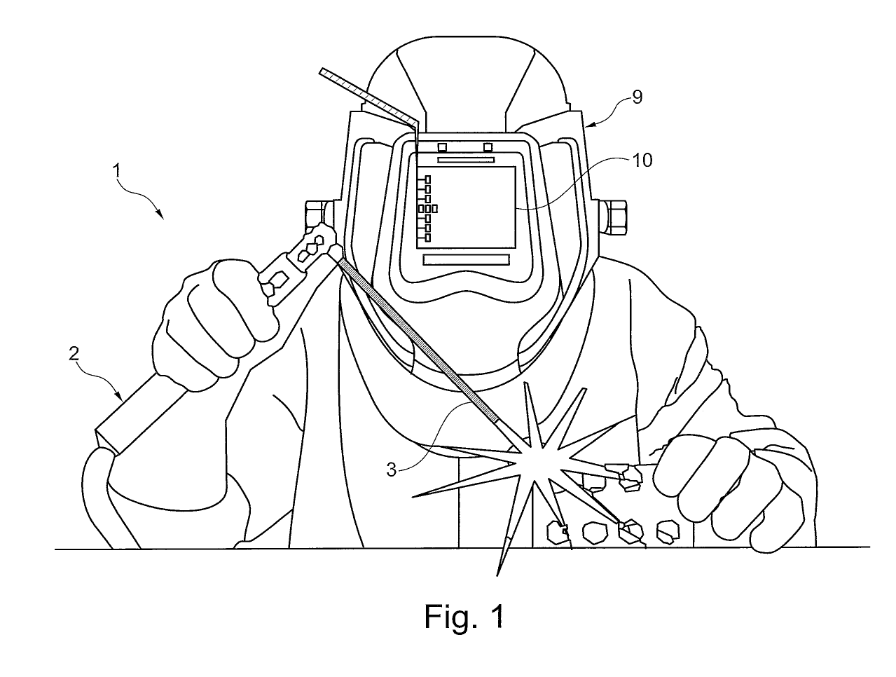

Optionally, the welding mask 9 comprises a visualization device 15. Such visualization device 15 is arranged to be easily visible by the welder during the welding process. As shown in FIGS. 1 and 8, the visualization device 15 is placed inside the welding mask 9, more particularly on one side of the darkened window 10.

With more detail, the visualization device 15 is configured to acquire the above mentioned velocity difference signal “Dw”, thus showing a representation of the velocity difference to the welder. Similarly, the visualization device 15 can be configured to acquire the voltage difference signal “Dv” mentioned above and to show a representation of the voltage difference to the welder. As shown schematically in FIG. 8, the visualization device comprises a plurality of LEDs 26. These leds are more particularly arranged in a cross, and are configured to lighten in such a way as to indicate whether the welder should go faster or slower, or if he should get nearer or farther from the weld area.

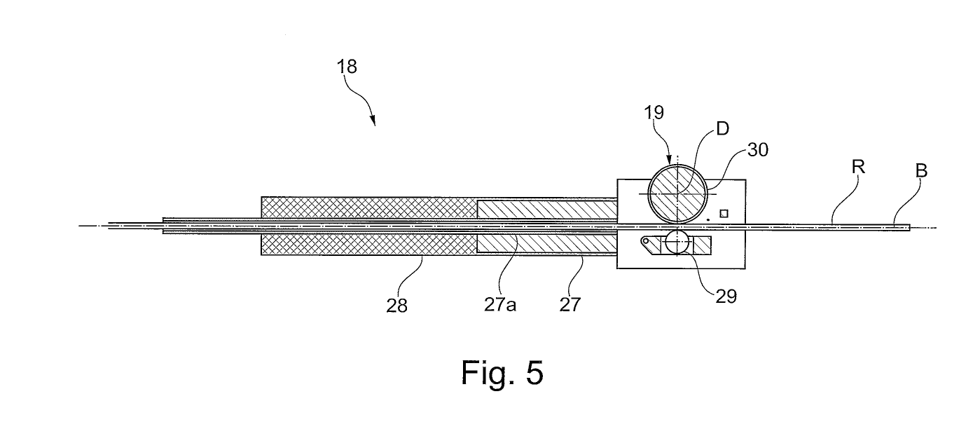

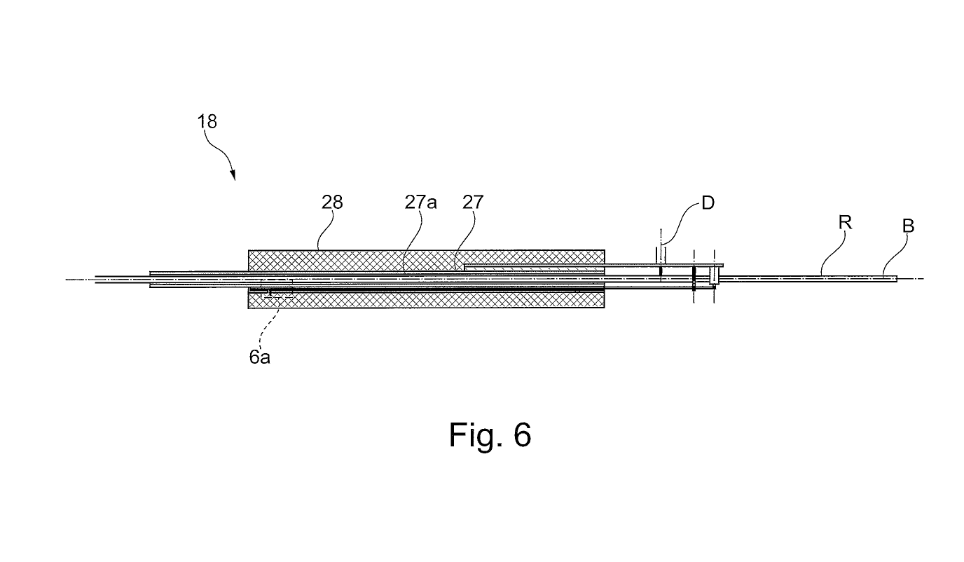

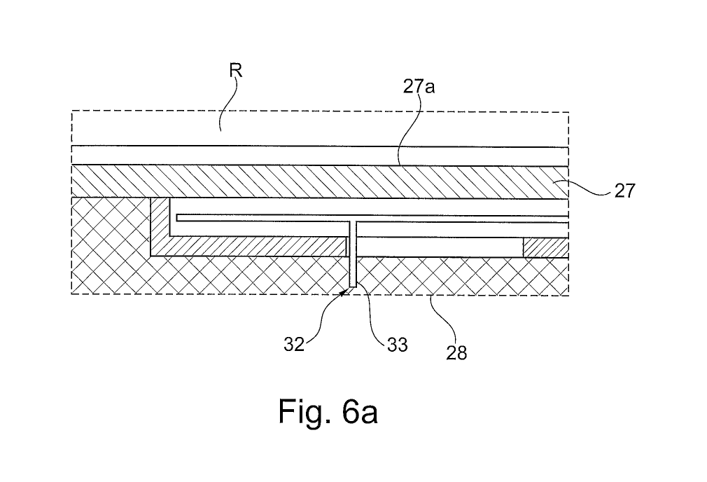

Referring specifically to FIGS. 5 and 6, the kit 1 can also comprises a handling apparatus 18 for a filler rod “R”. The handling apparatus 18 comprises a feeding device 19 configured to advance the filler rod “R” during welding.

With additional details, the handling apparatus 18 comprises a main body 27, configured to support the filler rod “R”. The main body 27 is more particularly axial-symmetric, and develops mainly along a longitudinal axis “B”. A handle 28 for the welder is attached to the main body 27. More particularly, the handle 28 surrounds the main body 27 of the handling apparatus 18.

The main body 27 has a central seat 27A in which the filler rod “R” is placed. As shown in FIG. 5, the handling apparatus 18 is provided with bearings 29, which are attached to the main body 27 and located in proximity of the central seat 27A, to support the filler rod “R” and allow it to move forward/backward. In other words, the filler rod “R” can move forward/backward inside the seat 27A by sliding on the bearings 29.

The feeding device 19 comprises a wheel 30 having a central axis “D” disposed transversally, and, in an embodiment, disposed perpendicularly to the longitudinal axis “B” of the main body 27.

In operation, the rim of the wheel 30 is in contact with the filler rod “R” so that it can be moved along the longitudinal axis “B” by a rotation of the wheel 30 along the central axis “D”. The feeding device 19 also comprises a motor 31. Such motor 31 is, in an embodiment, electric, more particularly electromagnetic, and is installed on the wheel 30 in order to actuate the filler rod “R”.

In an alternative embodiment, not shown in the drawings, the feeding device 19 comprises an electromagnetic actuation device for the filler rod “R” instead of the wheel 30 and the motor 31.

If the handling apparatus 18 is used, the processing module 8 may be configured to emit a feeding velocity signal “Sv” to the actuation module 14. The feeding velocity signal “Sv” is, in an embodiment, proportional to a feeding velocity value “Fv”. The actuation module 14 is therefore also configured to operate the feeding device 19 of the handling apparatus 18 as directed by the feeding velocity signal “Sv”.

Also, as shown in FIG. 6A, the handling apparatus 18 comprises a control interface 32 associated with the processing module 8. The control interface 32 is configured to emit a command signal “Cv” to the processing module 8, so that the welder can increase or decrease the feeding velocity signal “Sa”.

With additional detail, the control interface 32 comprises a button 33 placed on the handle 28. Specifically, the button 33 allows the welder to adjust the feeding velocity continuously; however the button 33 is designed as to give a tactile feedback in the form of “clicks” at predetermined intervals so that the welder can be made aware with a certain precision of the amount that the feeding velocity is being manually increased or decreased.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims

What is claimed is:

1. A welding assistance device comprising:
a welding mask;
a welding velocity sensor attached on the welding mask the welding velocity sensor being configured to detect a welding velocity; and
a visualization device attached to the welding mask and arranged to show a representation of the welding velocity and of consequent heat input to a welder.

2. The welding assistance device according to claim 1, wherein the welding mask comprises a darkened window for the welder, the visualization device being placed inside the welding mask, preferably on one side of the darkened window.

3. The welding assistance device according to claim 1 wherein the visualization device comprises a plurality of LEDs.

4. The welding assistance device according to claim 3, wherein the LEDS are arranged in a cross, the LEDs being configured to lighten in such a way as to give the welder an indication relating to a performance of a welding operation.

5. The welding assistance device according to claim 1 wherein the welding velocity sensor is configured to emit a welding velocity signal representing a value of the welding velocity; the welding assistance device further comprising a control unit comprising a data acquisition module configured to acquire the welding velocity signal, a processing module configured to compute a velocity difference between the welding velocity and a target velocity value for the welding velocity, the processing module being configured to emit a velocity difference signal representing the result of the velocity difference; the visualization device being configured to acquire the velocity difference signal and to show a representation of the velocity difference to a welder.

6. The welding assistance device according to claim 5, wherein the welding velocity sensor comprises a first optical sensor arranged to face the weld area and configured to detect a welding velocity relative to the first optical sensor; a reference frame sensor configured to detect a velocity of the reference frame sensor with respect to a fixed reference; a velocity computing module configured to compute the welding velocity as a difference between the velocities detected by the first optical sensor and the reference frame sensor.

7. The welding assistance device according to claim 6 wherein the reference frame sensor is a second optical sensor arranged to face the fixed reference.

8. The welding assistance device according to claim 1, wherein the processing module is configured to compute a voltage difference between a welding voltage and a target voltage, the processing module being configured to emit a voltage difference signal representing the result of the voltage difference; the visualization device being configured to acquire the voltage difference signal and to show a representation of the voltage difference to a welder.

9. An arc welding kit, comprising
a welding assistance device comprising:
a welding mask;
a welding velocity sensor attached on the welding mask, the welding velocity sensor being configured to detect a welding velocity and emit a welding velocity signal representing a value of the welding velocity;
a visualization device attached to the welding mask and arranged to show a representation of the welding velocity and of consequent heat input to a welder;
a control unit comprising a data acquisition module configured to acquire the welding velocity signal;
a processing module configured to compute a velocity difference between the welding velocity and a target velocity value for the welding velocity, the processing module being configured to emit a velocity difference signal representing the result of the velocity difference; the visualization device being configured to acquire the velocity difference signal and to show a representation of the velocity difference to a welder;
a welding tool configured to be held by a welder, the welding tool comprising an electrode, the welding tool comprising a voltage sensor configured to detect the welding voltage between the electrode and the weld area and to emit a voltage signal representing a value of the welding voltage.

10. The arc welding kit according to claim 9, wherein the electrode is consumable for performing a Shielded Metal Arc Welding weld.

11. The arc welding kit according to claim 9, wherein the electrode is non-consumable for performing a Tungsten Inert Gas weld.
